(19) United States
(12) Patent Application Publication
(10) Pub. No.: US 2026/0355017 A1
(43) Pub. Date: Nov. 12, 2026

(54) Safety Headwear Mounting System

(71) Applicant: Milwaukee Electric Tool Corporation, Brookfield, WI (US)

(72) Inventors: Andrea M. Schutz, Johnson Creek, WI (US); Nathaniel J. Peter, St Francis, WI (US); Rose M. Colacino, Milwaukee, WI (US); Craig A. Fluegge, Menomonee Falls, WI (US)

(21) Appl. No.: 19/629,645

(22) Filed: Mar. 26, 2026

Related U.S. Application Data
(60) Provisional application No. 63/801,361, filed on May 7, 2025.
(60) Provisional application No. 63/853,806, filed on Jul. 30, 2025.

(51) Int. Cl. A61F 9/06; A42B 3/18

(52) U.S. Cl. CPC A61F 9/06; A42B 3/18

(57) ABSTRACT

Various embodiments of a protective system include a hard hat or suspension system, a face shield, and a mounting component are provided. The mounting component allows for both fore/aft adjustment and pivoting of the face shield. The mounting component includes a rail and a slider which is movable along the rail. A pivot joint of the mounting component allows the face shield to pivot between a stowed position and an in use position. In specific embodiments, the pivot joint is positioned above the slider.

### CROSS-REFERENCE TO RELATED PATENT APPLICATION

The present application claims the benefit of and priority to U.S. Provisional Application No. 63/853,806, filed on Jul. 30, 2025, and to U.S. Provisional Application No. 63/801,361, filed on May 7, 2025, which are incorporated herein by reference in their entireties.

### BACKGROUND OF THE INVENTION

The present invention relates generally to the field of safety equipment. The present invention relates specifically to safety headwear accessories that provide face and vision protection. Workers may attach accessories to safety headwear, such as hard hats or helmets and/or headgear, such as a suspension system. The safety headwear may include coupling mechanisms, such as coupling slots at varying locations. Further, in some situations face shields or welding shields may provide useful protection and in other circumstances the face shields or welding shields may be unnecessary and can be moved into a retracted position away from the wearer's face using a mounting system.

### SUMMARY OF THE INVENTION

One embodiment of the invention relates to a protective system. The protective system includes a face shield and a hard hat or suspension system configured to support the face shield. The protective system includes a mounting component configured to coupled to the hard hat or the suspension system and support the face shield relative to the hard hat or the suspension system. The mounting component includes a rail and a pivot joint. The rail defines a longitudinal axis and includes one or more projections. The pivot joint is configured to move the face shield between a stowed an in-use position. the pivot joint includes a slider configured to move along the longitudinal axis of the rail to adjust a position of the face shield. When the button is actuated, the latch is pivoted to a position spaced from the one or more projecting portions of the rail such that the position of the slider is movable along the longitudinal axis of the rail. When the button is not engaged, the latch engages the one or more projecting portions to resist movement of the slider along the longitudinal axis.

Another embodiment of the invention relates to a protective system. The protective system includes a face shield and a hard hat. A mounting component is configured to support the face shield relative to the hard hat. The mounting component includes a rail defining a longitudinal axis and including one or more protrusions and a pivot joint. The pivot joint is configured to move the face shield between a stowed position and an in-use position. The pivot joint includes a latch. The latch is pivotable between a disengaged position in which the latch is spaced from the one or more protrusions of the rail and an engaged position in which the latch interfaces with the one or more protrusions to secure the pivot joint to the rail.

Another embodiment of the invention relates to a protective system. The protective system includes a face shield and a hard hat. A mounting component is configured to support the face shield relative to the hard hat. The mounting component includes a rail defining a length along a longitudinal axis. The rail includes at least one protrusion extending outward from the rail and a clip configured to engage a slot defined in the hard hat. The mounting component further includes a pivot joint configured to move the face shield between a stowed position and an in-use position. The pivot joint includes a button and a latch connected to the button and configured to be actuated by the button. The latch is actuated between a first position in which the latch is disengaged from the at least one protrusion such that the pivot joint is adjustable and a second position in which the latch is engaged with the at least one protrusion such that the pivot joint is secured.

Additional features and advantages will be set forth in the detailed description which follows, and will be readily apparent to those skilled in the art from the description or recognized by practicing the embodiments as described in the written description and/or shown in the accompany drawings. It is to be understood that both the foregoing general description and the following detailed description are exemplary.

The accompanying drawings are included to provide further understanding and are incorporated in and constitute a part of this specification. The drawings illustrate one or more embodiments and, together with the description, serve to explain principles and operation of the various embodiments.

### BRIEF DESCRIPTION OF THE DRAWINGS

This application will become more fully understood from the following detailed description, taken in conjunction with the accompanying figures, wherein like reference numerals refer to like elements in which:

FIG. 1A is a perspective view of a protective system for safety headwear, according to an exemplary embodiment.

FIG. 1B is a side view of a protective system for safety headwear with a welding face shield in a forward, in use position, according to an exemplary embodiment.

FIG. 1C is a side view of the protective system for safety headwear of FIG. 1B with the welding face shield in a forward, stowed position, according to an exemplary embodiment.

FIG. 1D is a side view of the protective system for safety headwear with the welding face shield in a rear, in use position, according to an exemplary embodiment.

FIG. 1E is a side view of the protective system for safety headwear of FIG. 1D with the welding face shield in a rear, stowed position, according to an exemplary embodiment.

FIG. 2 is a front perspective view of a mounting component of the protective system of FIG. 1, according to an exemplary embodiment.

FIG. 3 is a rear perspective view of the mounting component of the protective system of FIG. 1, according to an exemplary embodiment.

FIG. 4 is a right side view of the mounting component of FIG. 2, according to an exemplary embodiment.

FIG. 5 is a left side view of the mounting component of FIG. 2, according to an exemplary embodiment.

FIG. 6 is an exploded view of the mounting component of FIG. 2, according to an exemplary embodiment.

FIG. 7 is a perspective view of the mounting component of FIG. 2, according to an exemplary embodiment.

FIG. 8 is a cross-sectional view of the mounting component in an engaged position taken along line 8—8 in FIG. 5, according to an exemplary embodiment.

FIG. 9 is a cross-sectional view of the component in a disengaged position, according to an exemplary embodiment.

FIG. 10 is a rear view of a portion of the mounting component, according to an exemplary embodiment.

FIG. 11 is a side view of a protective system for safety headwear with a welding face shield in a forward, in use position, according to another exemplary embodiment.

FIG. 12 is a side view of the protective system for safety headwear of FIG. 11 with the welding face shield in a forward, stowed position, according to an exemplary embodiment.

FIG. 13 is a front perspective view of a mounting component of the protective system of FIG. 11, according to an exemplary embodiment.

FIG. 14. is a rear perspective view of the mounting component of the protective system of FIG. 11, according to an exemplary embodiment.

FIG. 15 is a right side view of the mounting component of FIG. 13, according to an exemplary embodiment.

FIG. 16 is a left side view of the mounting component of FIG. 13, according to an exemplary embodiment.

FIG. 17 is a cross-sectional view of the mounting component in an engaged position taken along line 17—17 in FIG. 16, according to an exemplary embodiment.

FIG. 18 is a cross-sectional view of the mounting component in a disengaged position taken along line 8—8 in FIG. 5, according to an exemplary embodiment.

FIG. 19 is a front perspective view of a mounting component for use with a protective system, according to an exemplary embodiment.

FIG. 20. is a rear perspective view of the mounting component, according to an exemplary embodiment.

FIG. 21 is a right side view of the mounting component of FIG. 19, according to an exemplary embodiment.

FIG. 22 is a left side view of the mounting component of FIG. 19, according to an exemplary embodiment.

FIG. 23 is a left side view of the mounting component in a disengaged position, according to an exemplary embodiment.

FIG. 24 is an exploded view of the mounting component of FIG. 19, according to an exemplary embodiment.

FIG. 25 is a rear perspective view of a rail of the mounting component, according to an exemplary embodiment.

FIG. 26 is a cross-sectional view of the mounting component in an engaged position taken along line 26—26 in FIG. 22, according to an exemplary embodiment.

### DETAILED DESCRIPTION

Referring generally to the figures, various embodiments of a protective system are shown. Various embodiments of the protective system include a protective work/construction helmet, shown as a hard hat and a protective accessory such as a face shield, shown as a welding face shield. In specific embodiments, the protective system includes a welding face shield and headgear (e.g., a suspension system). The protective system includes a mounting component that can be coupled to the hard hat or the headgear interchangeably. Applicant believes the interchangeable mounting component allows for quick and easy customization of the protective system to particular work environments improving efficiency on the work site.

In various embodiments, the mounting component includes both fore/aft adjustment and pivoting adjustment. In specific embodiments, the fore/aft adjustment of the mounting component includes a rail and a slider allowing for sliding along the rail. In a specific embodiment, the fore/aft adjustment mechanism includes a biased latch that is actuated by a button to engage and/or disengage with a row of protrusions extending from the rail that engage and secure the latch in place along the rail. In various embodiments, the slider can be disengaged from the rail to allow for quick and easy removal of the face shield.

In various embodiments, the pivot joint is integrated with and positioned above the slider. As will be generally understood, the pivot joint allows the welding face shield to pivot up (e.g., into stowed position) and pivot down (e.g., into use position). In specific embodiments, an outer portion of the button structure is rotatable such that the pivot joint can be tightened or loosened relative to the rail and/or fore/aft adjustment mechanism. In specific embodiments, the pivot joint includes a locking feature configured to secure the welding face shield in the upper/stowed position and a lower stop configured to prevent further downward pivot of the welding face shield when it is in the down/in use position. Applicant believes the combination of pivot joint and the rail and slider improves safety and efficiency by ensuring a correct and secure fit for various users and helmets of different sizes that can be adjusted as necessary for various work environments. Furthermore, the mounting component improves safety on a work site by allowing a user to make fore/aft adjustments of the face shield, fully removing the face shield, and remounting of the face shield all without needing to remove the helmet or hard hat.

In various embodiments, the fore/aft adjustment of the mounting component includes a rail and an arm allowing for positioning or clipping the arm along the rail. In a specific embodiment, the fore/aft adjustment mechanism includes a biased latch that is actuated by a button to engage and/or disengage with a bottom edge of the rail that together with the clipping arm secures the latch in place along the rail. In various specific embodiments, the arm can be disengaged from the rail to allow for quick and easy removal of the face shield. In contrast to the other fore/aft movement discussed herein, the arm of the mounting component allows flexibility in locating a desired position along the rail and clipping or positioning the mounting component at that mounting location. In specific embodiments, the movement is vertical, meaning the mounting component moves upward, away from rail when disengaged and downward toward the rail when a mounting location has been chosen.

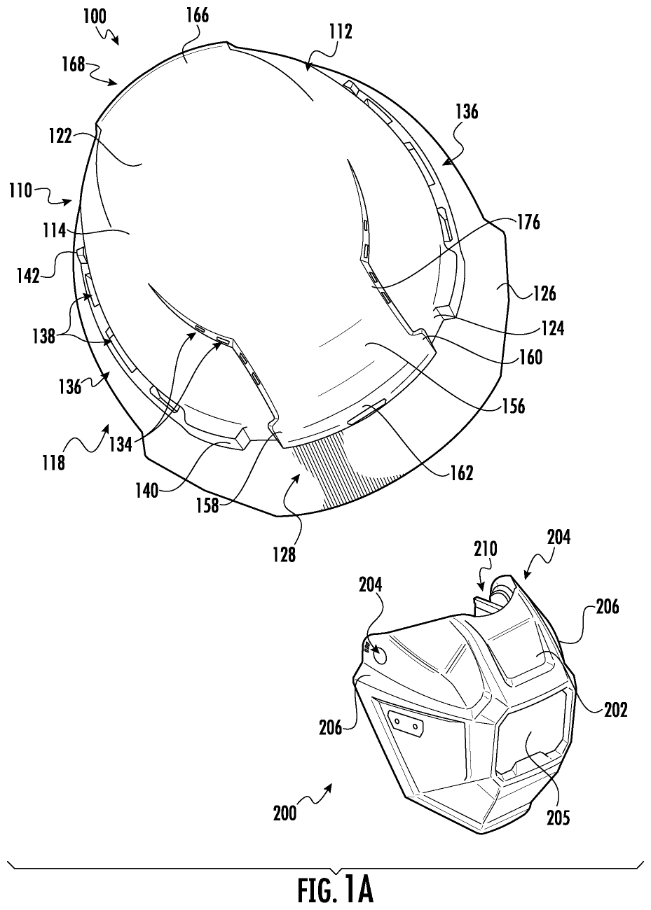

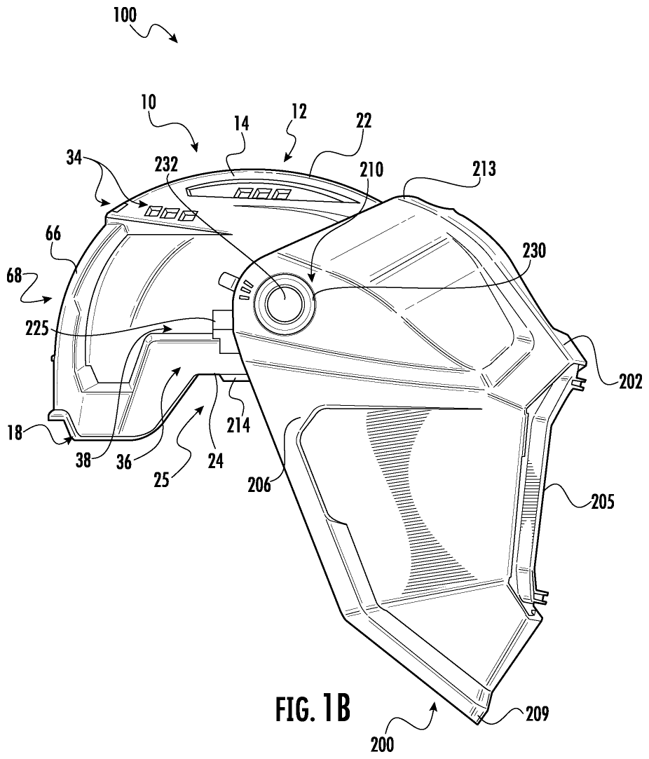

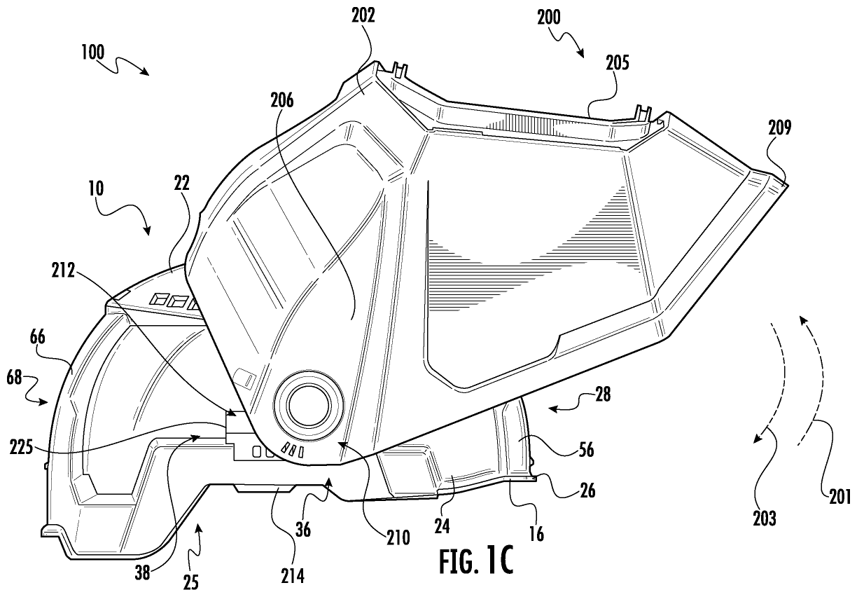

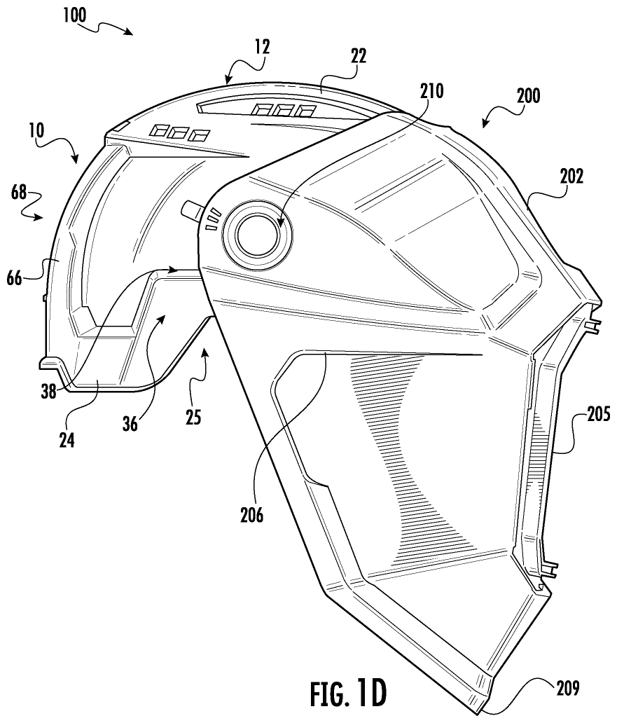

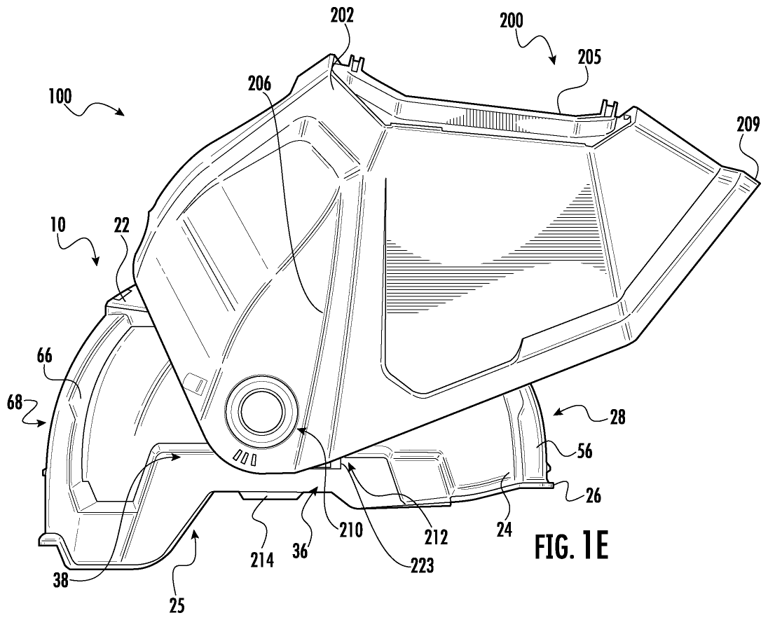

Referring to FIG. 1A, a perspective view of a protective system 100 including a work or construction helmet, shown as a hard hat is shown according to an exemplary embodiment. In various embodiments, protective system 100 includes a construction helmet, shown as hard hat 110. Hard hat 110 includes an outer shell 112. Outer shell 112 includes an exterior surface 114 and an interior surface that defines a cavity 118 sized to receive a head of a user and/or wearer. Outer shell 112 includes a crown portion 122 and a bottom segment 124 defining a lower circumference of hard hat 110. In various embodiments, a brim 126 extends radially outward from a portion of the lower circumference.

In a specific embodiment, brim 126 extends radially outward from the front 128 of hard hat 10 (see e.g., FIG. 1B) and specifically outer shell 112. In specific embodiments, outer shell 112 further includes a plurality of apertures or vents 134. Vents 134 extend through outer shell 112 providing fluid communication between cavity 118 and the ambient air proximate to exterior surface 114 of outer shell 112.

Various embodiments include one or more mounting ridges configured to couple to and/or support hard hat accessories. A side accessory support ridge or auxiliary mounting ridge 136 is positioned on a lateral side of outer shell 112 along the bottom segment 124. In various specific embodiments, an additional side accessory support ridge or auxiliary mounting ridge 136 is positioned on an opposing lateral side of outer shell 112 along the bottom segment 124. In other words, in various embodiments, hard hat 110 includes a pair of auxiliary mounting ridges 136 positioned on respective lateral sides of outer shell 112.

Auxiliary mounting ridge 136 includes a first end 140 and a second end 142 opposing the first end 140. A plurality of apertures or slots 138 are positioned along auxiliary mounting ridge 136 between the first end 140 and the second end 142. Slots 138 are configured to receive a coupling mechanism, such as clips, or a portion of a hard hat accessory to couple the accessory to outer shell 112. In a specific embodiment, auxiliary mounting ridge 136 includes three slots 138 positioned between the first end 140 and the second end 142. Auxiliary mounting ridge 136 supports accessories for hard hat 10, such as face shields, welding shields, ear muffs, tool or eyeglass holders, lamp supports, face shields, and/or reflectors, etc.

Hard hat 110 further includes a front mounting ridge 156 positioned on the front 128 of outer shell 112. Front mounting ridge 156 includes a first edge 158 protruding outwardly from the front mounting ridge 156 and a second edge 160 opposing the first edge 158 and protruding outwardly from the front mounting ridge 156. In a specific embodiment, front mounting ridge 156 includes a detent 162 positioned between the first edge 158 and the second edge 160. The detent 162 is configured to interface with an accessory and/or a mounting bracket for an accessory. The first edge 158 and second edge 160 of the front mounting ridge 156 each extend toward the bottom segment 124 of the outer shell 112. The first edge 158 and second edge 160 each include a portion with an increased width as the first edge 158 and second edge 160 approach the bottom segment 124 and/or the brim 126.

In various embodiments, the hard hat 110 includes a second or rear mounting ridge 166 located along at the rear 168 of the hard hat 110. This allows a user to attach accessories and/or accessory mounting brackets to both the front 128 and rear 168 of hard hat 110. Rear mounting ridge 166 is substantially the same as front mounting ridge 156.

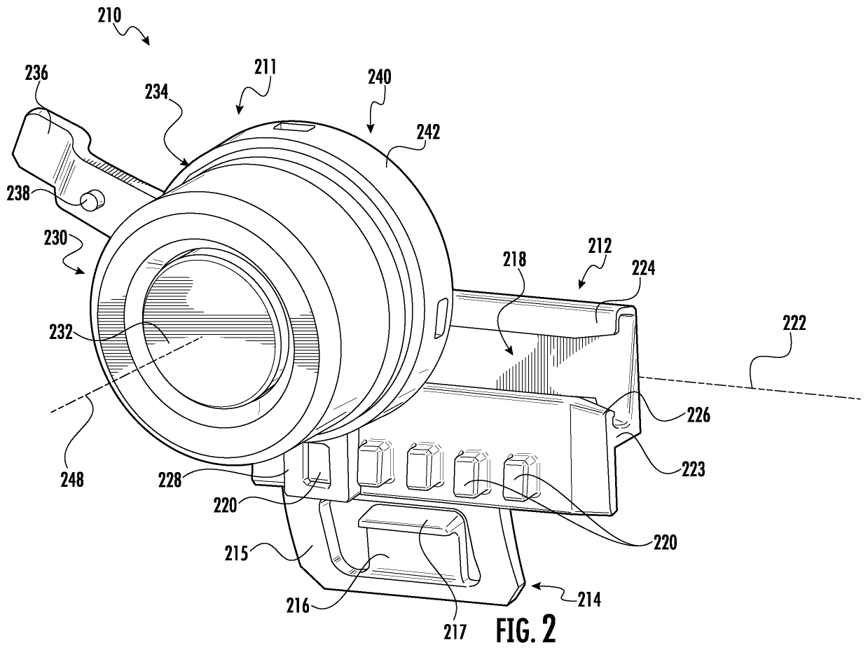

In various embodiments, protective system 100 includes an accessory, shown as a face shield and specifically a welding face shield 200. Welding face shield 200 includes a body 202 with one or more bores 204. Each of the one or more bores 204 is configured to engage a mounting component (see e.g., 210 in FIG. 2). In a specific embodiment, welding face shield 200 includes a pair of bores 204 defined in opposing lateral portions 206 of body 202. In a specific embodiment, welding face shield 200 includes a lens 205 positioned on a front or middle portion of body 202 in between opposing lateral portions 206. In specific embodiments, the lens 205 is a passive lens (e.g., the lens is dark to protect user vision). In specific embodiments, the lens 205 is an auto-darkening lens. As will generally be understood, an auto-darkening lens darkens when welding arc/bright light etc. is detected.

Referring to FIGS. 1B-E, side views of protective system 100 including a work or construction helmet, shown as a hard hat 10 are shown according to an exemplary embodiment. As will be generally understood, various types of helmets, hard hats, safety helmets with brims, safety helmets without brims, etc. can be utilized with welding face shield 200 and/or mounting component 210 to form protective system 100. Hard hat 10 is substantially the same as hard hat 110 except for the differences discussed herein.

Hard hat 10 includes an outer shell 12. Outer shell 12 includes an exterior surface 14 and an interior surface 16. Interior surface 16 defines a cavity 18. Outer shell 12 includes a crown portion 22 and a bottom segment 24 defining a lower circumference of hard hat 10. In various embodiments, a lip 26 defines a forwardmost portion of bottom segment 24. Lip 26 has size and/or dimension significantly less than a brim 26. In other words, lip 26 is a shorter brim. In various embodiments, lip 26 extends less than a maximum distance outward from exterior surface 14 of outer shell 12. In a specific embodiment, lip 26 extends less than 10 mm from exterior surface 14, specifically less than 9 mm, and more specifically less than 8.55 mm. In a specific embodiment, the lip 26 extends about 8.17 mm from exterior surface 14 of hard hat 10.

Outer shell 12 further includes recess 25 positioned between front 28 and rear 68 of hard hat 10 proximate to the ears of a wearer. In a specific embodiment, recess 25 extends a height above a portion of bottom segment 24 proximate to front 28 and/or rear 68 of hard hat 10.

A side accessory support ridge or auxiliary mounting ridge 36 is positioned on a lateral side of outer shell 12 along the bottom segment 24. A plurality of apertures or slots 38 are positioned along auxiliary mounting ridge 36 between a first end and a second end. Slots 38 are configured to receive an accessory or a coupling mechanism, such as mounting component 210 that supports and couples welding face shield 200 to outer shell 12 and/or hard hat 10. In various embodiments, an additional auxiliary mounting ridge 36 is positioned on an opposing lateral side of outer shell 12 along the bottom segment 24. Hard hat 10 further includes a front mounting ridge 56 positioned on the front 28 of outer shell 12. In various embodiments, the hard hat 10 includes a second or rear mounting ridge 66 located along at the rear 68 of the hard hat 10. This allows a user to attach accessories and/or accessory mounting brackets to both the front 28 and rear 68 of hard hat 10. Rear mounting ridge 66 is substantially the same as front mounting ridge 56.

Referring to FIG. 1B, details of protective system 100 and specifically welding face shield 200 in the in use position are shown. When welding face shield 200 is in use, body 202 extends downward in front of hard hat 10 and is positioned in front of front mounting ridge 56 and a face of the wearer. Further, as will be discussed in greater detail below, welding face shield 200 is moveable fore/aft relative to hard hat 10 and/or auxiliary mounting ridge 36. As shown in FIG. 1B, welding face 200 is positioned in a forward position relative to mounting component 210.

As can be seen, clip 214 is positioned within a slot 38 of auxiliary mounting ridge 36. In specific embodiments, protective system 100 includes a pair of mounting components 210 such that a pair of clips 214 are positioned within a pair of auxiliary mounting ridges 36. In specific embodiments, auxiliary mounting ridge 36 includes three slots 38 and clip 214 is received within a middle or central slot 38. In other embodiments, clip 214 of mounting component 210 can be positioned within and/or received by a different slot 38.

Rail 212 of mounting component 210 extends between a first or forward edge 223 (see e.g., FIGS. 1E and 2) and a second or rear edge 225. When welding face shield 200 is in a forward position (i.e. in use or stowed), second or rear edge 225 extends beyond a rearmost portion of welding face shield 200 and in particular lateral portion 206 of welding face shield 200.

As shown in FIGS. 1B-E, protective system 100 is fully assembled, including a hard hat 10, 110, welding face shield 200 and mounting component(s) 210. When mounting component 210 is coupled to welding face shield 200, at least a portion of mounting component 210 is positioned outward from, such as radially or laterally outward from welding face shield body 202. In various specific embodiments, a knob 230 and/or button 232 are positioned along an exterior surface of lateral portion 206. In other words, while portions of mounting component 210 are blocked by welding face shield 200, knob 230 and button 232 are accessible to a user. In various embodiments, welding face shield 200 and/or lateral portion 206 are positioned between knob 230 and button 232 of mounting component 210 and hard hat 10.

Referring to FIG. 1C, details of protective system 100 and specifically welding face shield 200 is in an in stowed position are shown according to an exemplary embodiment. Further, as shown in FIG. 1C, welding face shield 200 is positioned in a forward position relative to mounting component 210.

When a user is done using welding face shield 200, welding face shield can be moved from the in use position (see e.g., FIG. 1B) to the stowed position in which welding face shield 200 is positioned above bottom segment 24 of hard hat 10. In the stowed position an upper edge 213 of welding face shield 200 is positioned along crown 22 of hard hat 10. Further, in the stowed position, a user can access front mounting ridge 56. In other words, in the stowed position, welding face shield 200 does not block access to front mounting ridge 56 of hard hat 10.

For such a position change, welding face shield 200 is moved in a first direction, shown by arrow 201. In specific embodiments, the first direction is counterclockwise. When a user is ready to restart work, welding face shield 200 can be moved in a second direction, shown by arrow 203. The second direction is opposite the first direction. In specific embodiments, the second direction is clockwise.

Referring to FIG. 1D, details of protective system 100 and specifically welding face shield 200 is in an in use position are shown. As shown in FIG. 1D, welding face 200 is positioned in a rear position relative to mounting component 210. Clip 214 is positioned within slot 38 of auxiliary mounting ridge 36 and specifically within a middle or central slot 38 (see e.g., FIG. 1E). However, in the rear position, clip 214 and rail 212 are positioned entirely behind welding faces shield 200 and/or lateral portion 206. In other words, rail 212 and clip 214 are not visible in such a position.

Referring to FIG. 1E, details of protective system 100 and specifically welding face shield 200 is in an in stowed position are shown. Further, as shown in FIG. 1E, welding face 200 is positioned in the rear position due to mounting component 210. When welding face shield 200 is in a rear, stowed position, first or front edge 223 of rail 212 is visible.

In the stowed positions (see e.g., FIGS. 1C and 1E), a lowermost edge 209 of welding face shield 200 is positioned above front mounting ridge 56 of hard hat 10. In a specific embodiment, the lowermost edge 209 of welding face shield 200 is positioned above the crown 22 of hard hat 10. In the stowed position, a user can access front mounting ridge 56 because welding face shield 200 does not block front mounting ridge 56 of hard hat 10.

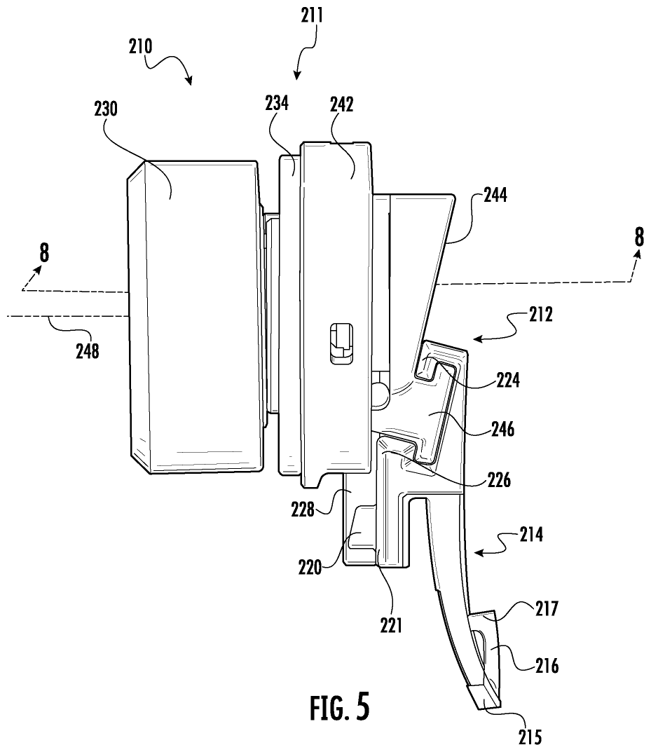

Referring to FIGS. 2-5, details of a mounting component 210 that can be utilized with protective system 100 are shown according to an exemplary embodiment. Mounting component 210 can be utilized with hard hat 110 to support an accessory such as a welding face shield 200. In specific embodiments, a pair of mounting components 210 are couplable to hard hat 110 and welding face shield 200. Similarly, mounting component 210 can be utilized with headgear and/or a suspension system to support an accessory such as welding face shield 200. In specific embodiments, the rail 212 is integrated with the headgear and/or suspension. In other words, the rail 212 and/or mounting component 210 is not removable from the headgear and/or suspension.

In various embodiments, mounting component 210 includes a rail 212 and clip 214. In specific embodiments, rail 212 is integrally formed with clip 214. Clip 214 is configured to be coupled to hard hat 110 and/or a suspension system. When clip 214 is coupled to hard hat 110, clip 214 is received within a slot 138 of auxiliary mounting ridge 136. In various embodiments, clip 214 includes a clip body 215 extending downward (in the orientation shown in FIG. 2) and away from rail 212. A tab, shown as resilient tab 216 extends from body 215 toward rail 212. An engagement surface, shown as an upward facing surface 217 is positioned on an uppermost portion of resilient tab 216. When clip 214 is positioned within a slot 138, resilient tab 216 and/or upward facing surface 217 engages a lower edge and/or downward facing surface of auxiliary mounting ridge 136.

As will be generally understood, rail 212 allows for fore/aft movement of accessories such as welding face shield 200. Rail 212 includes a sliding channel 218 defined between a first retaining arm 224 and a second retaining arm 226. Sliding channel 218 extends along a longitudinal axis 222 of rail 212. In other words, a major axis of sliding channel 218 is aligned and/or has the same orientation as a major axis of mounting component 210. In a specific embodiment, sliding channel 218 forms a T-shape. In other words, first retaining arm 224, second retaining arm 226 and an interior surface of sliding channel 218 together define a T-shaped space.

Rail 212 further includes an exterior surface 221 that faces away from sliding channel 218. In various embodiments, one or more projecting portions or protrusions 220 extend outward from exterior surface 221. In various embodiments, at least on protrusion 220 extends outward from exterior surface 221. In specific embodiments, rail 212 includes a row of protrusions 220 that are aligned along longitudinal axis 222. In a specific embodiment, rail 212 includes five protrusions 220 providing five positions for forward and/or aft movement of mounting component 210 and/or welding face shield 200. In other embodiments, a different number of protrusions 220 (e.g., 3, 4, 6, 7, etc.) can be utilized. The number of protrusions 220 corresponds to the number of positions a pivot joint 211 and specifically a latch 228 can be secured along a length of rail 212.

A slider 240 is removably couplable to pivot joint 211 and configured to engage rail 212. Slider 240 includes a rear surface 244 that faces rail 212 when slider 240 is engaged with rail 212. Slider 240 includes a projection 246 extending from rear surface 244 and toward rail 212. In a specific embodiment, projection 246 has a T-shaped cross-section.

Pivot joint 211 is configured to allow welding face shield 200 to move and/or pivot between a up or stowed position (e.g., above brim 126 of hard hat 110) and a down or in use position in which the welding face shield 200 is positioned in front of hard hat 110 and a user's face. In various embodiments, pivot joint 211 is integral with rail 212. In specific embodiments, pivot joint 211 is integral with slider 240 and positioned above rail 212 and clip 214. Pivot joint 211 includes a latch 228 that is configured to engage rail 212 and specifically protrusions 220 of rail 212. A bore 256 of latch 228 engages and surrounds a protrusion 220 when latch 228 is secured to rail 212. In other words, latch 228 is positioned around a perimeter of the protrusion 220 when the latch 228 is engaged with the protrusion 220. As will be discussed in greater detail below, latch 228 is a biased latch. In specific embodiments, latch 228 is a spring biased latch.

Mounting component 210 further includes a knob 230. Knob 230 is configured to turn or rotate about axis of rotation 248. When knob 230 is rotated in a first direction, pivot joint 211 is tightened relative to rail 212 and outer shell 112 when mounting component 210 is coupled to hard hat 110. As will be generally understood, when knob 230 is tightened, a force required to lift and lower welding face shield 200 is increased. Knob 230 is rotatable in a second direction, opposite the first direction. When knob 230 is rotated in the second direction, pivot joint 211 is loosened relative to rail 212 and outer shell 112 when mounting component 210 is coupled to hard hat 110. As knob 230 is loosened, the force required to lift and lower welding face shield 200 is decreased.

An actuator, shown as button 232 is positioned within knob 230. In other words, button 232 surrounds, such as radially surrounds the button 232. Button 232 is configured to engage latch 228 and move latch 228 between an engaged and disengaged position with rail 212 and in particular protrusions 220. Mounting component 210 further includes an adjuster 234. Adjuster 234 is configured to engage and change an angle of the body 202 of the face shield 200. In various embodiments, adjuster 234 includes a projecting arm 236 that extends away from axis of rotation 248. In a specific embodiment, arm 236 includes an arm projection 238 extending outward and away from rail 212. Arm projection 238 is configured to engage and/or connect to welding face shield 200 and/or body 202 of face shield 200.

In various embodiments, mounting component 210 includes a pivot lock to secure welding face shield 200 in the up or stowed position (e.g., above brim 126 of hard hat 110) and the down or in use position (e.g., the welding face shield 200 is positioned in front of hard hat 110 and a user's face). The pivot lock includes lock 242 positioned between slider 240 and adjuster 234. Lock 242 is coupled to slider 240 which is positioned between lock 242 and rail 212.

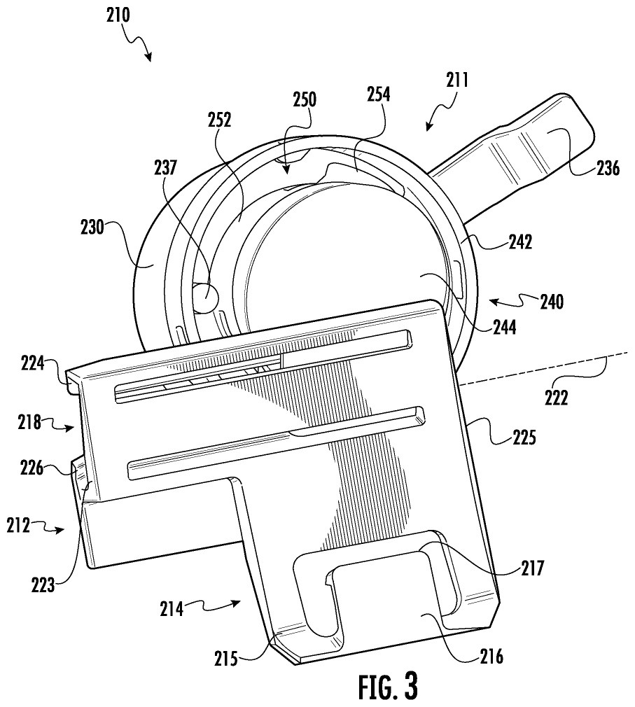

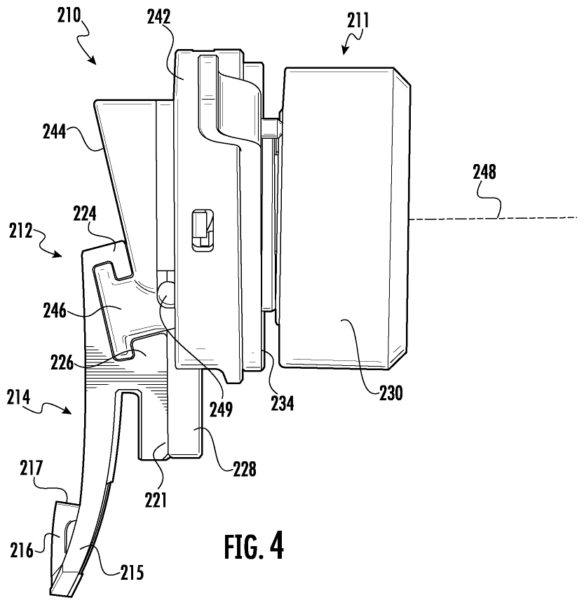

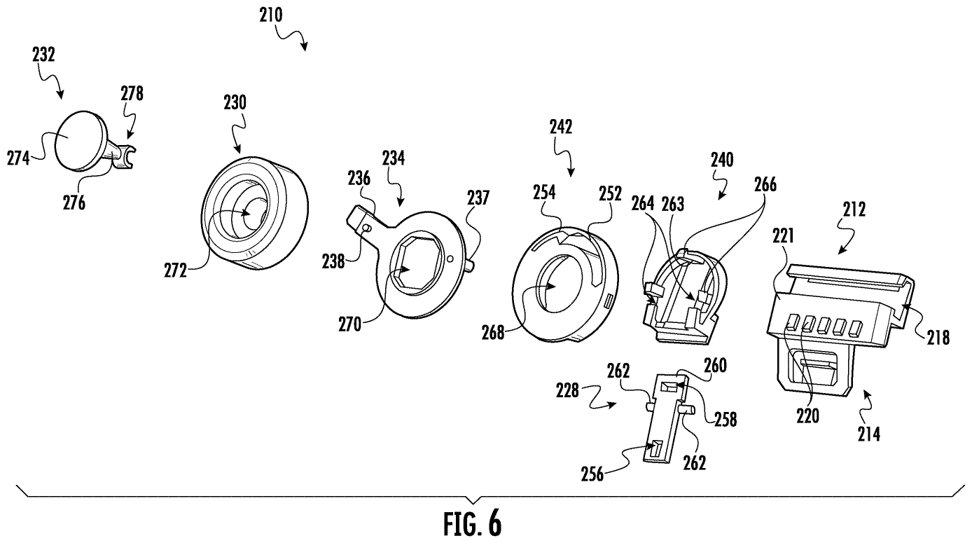

Referring to FIG. 6, an exploded view of mounting component 210 is shown according to an exemplary embodiment. A first end of latch 228 includes bore 256 that is configured to engage rail 212 and specifically one of projections 220 to secure pivot joint 211 to rail 212 of mounting component 210. A second end of latch 228 includes a second bore 258 that is positioned between an upper portion 260 of latch 228 and bore 256. Second bore 258 and/or upper portion 260 are configured to engage button 232. Latch 228 further includes first and second connectors 262 that extend laterally outward from latch 228. Connectors 262 are configured to be received within a pair of recesses 264 defined along a front surface 263 of slider 240. The front surface 263 of slider 240 opposes rear surface 244 of slider 240. When mounting component 210 is assembled, each connector 262 is positioned in a corresponding recess 264 and movable within that recess 264 about latch pivot axis 249 (see e.g., FIG. 4) as latch 228 is pivoted to engage or disengage from rail 212.

Front surface 263 of slider 240 further includes one or more tabs 266 that are received within and engage lock 242. Lock 242 includes a central bore 268 that receives a portion of button 232 when mounting component 210 is assembled. Lock 242 further includes a locking channel 250 that extends in a circumferential direction and is positioned above central bore 268. Locking channel 250 includes a first section 252 that has a first dimension and a second section 254 that has a second dimension. In specific embodiments, the second dimension is less than the first dimension. In various embodiments, the first and second dimensions are widths defined between opposing edges of locking channel 250.

Adjuster 234 further includes a bore shown as central bore 270 that receives a portion of button 232 when mounting component 210 is assembled. Adjuster 234 includes a rearward extending projection 237 (e.g., extends away from knob 230). As will be discussed in greater detail below, rearward extending projection 237 is received within locking channel 250 and is configured to secure welding face shield 200 in place (e.g., stowed or in use positions).

Knob 230 includes a bore, shown as a central bore 272 that is configured to receive a portion of button 232 when mounting component 210 is assembled. Button 232 includes an engagement or actuation surface 274, a body 276 that extends rearward (e.g., toward rail 212) through knob 230, adjuster 234, and lock 242. Button 232 further includes a clip 278 on an end opposing actuation surface 274.

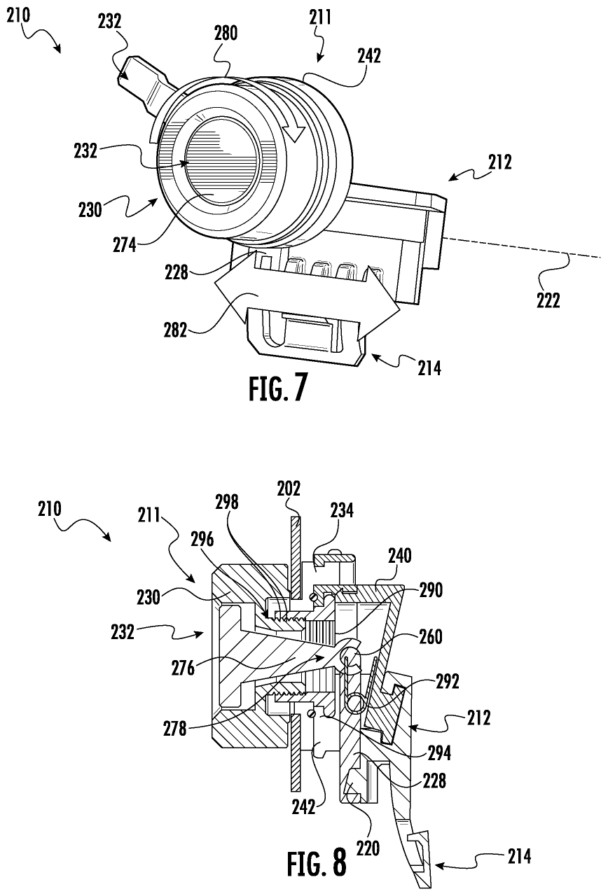

Referring to FIGS. 7-9, details of the operation of mounting component 210 are shown, according to an exemplary embodiment. As previously discussed, mounting component 210 is movable fore/aft (e.g., along lateral sides of hard hat 110 and/or suspension system). Arrow 282 shows the direction of movement of when welding face shield 200 is moved fore/aft. Arrow 282 and the fore/aft movement of pivot joint 211 and/or welding face shield 200 happens along a length of rail 212 and/or major or longitudinal axis 222. When protective system 100 is assembled, body 202 of welding face shield 200 is positioned between knob 230 and adjuster 234.

Knob 230 is rotatable in first direction 280 which shows the tightening direction of knob 230. A sealing device, shown as O-ring 294 is positioned between adjuster 234 and lock 242. When knob 230 is turned, compression of O-ring 294 is changed. Knob includes a rear connector 296. In specific embodiments, rear connector 296 includes threads 298. Rear connector 296 is configured to engage a nut shown as flanged nut 290. Flanged nut 290 is positioned within lock 242.

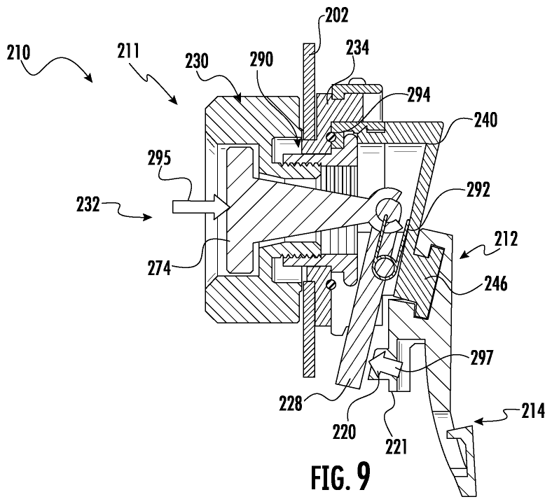

In various embodiments, latch 228 is biased by a spring. In specific embodiments, latch 228 is biased by a torsion spring 292. As shown in FIG. 8, button 232 and specifically actuation surface 274 is recessed relative to an outermost surface of knob 230. As shown in FIG. 9, when button 232 and specifically actuation surface 274 is engaged as shown by arrow 295, actuation surface 274 is moved further into knob 230 such that a depth of the recession of button 232 is increased.

When button 232 is depressed (see e.g., arrow 295), clip 278 that is coupled to latch 228 pivots the latch 228 such that bore 256 is removed from the protrusion 220. As shown by arrow 297, the lower portion of latch 228 moves from being positioned along exterior surface 221 of rail 212 to a position in which latch 228 is spaced apart from rail 212 and exterior surface 221. When latch 228 is pivoted into a disengaged position (see e.g., FIG. 9), slider 240 can freely move along rail 212 to adjust the position of welding face shield 200. When button 232 is released, latch 228 is engaged with a protrusion 220 (see e.g., FIG. 8) such that protrusion 220 resists fore/aft movement and the position of welding face shield 200 is secured.

Referring to FIG. 10, details of the locking mechanism of pivot joint 211 is shown according to an exemplary embodiment. As previously discussed, mounting component 210 includes a pivot lock that secures welding face shield 200 in the up or stowed position (e.g., above brim 126 of hard hat 110) and the down or in use position (e.g., the welding face shield 200 is positioned in front of hard hat 110 and a user's face).

When rearward extending projection 237 is positioned within first section 252 of locking channel 250 and engaged with lower edge 286, welding face shield 200 cannot be moved lower. In other words, lower edge 286 acts as a lower stop for welding face shield 200 that secures welding face shield in the in use position. When a user pivots welding face shield 200 into an up or stowed position, rearward extending projection 237 moves through locking channel 250 as shown by arrow 284 in a direction toward second section 254 of locking channel 250. When rearward extending projection 237 reaches upper edge 288, welding face shield 200 cannot be moved higher or further up. In other words, upper edge 288 acts as an upper stop for welding face shield 200.

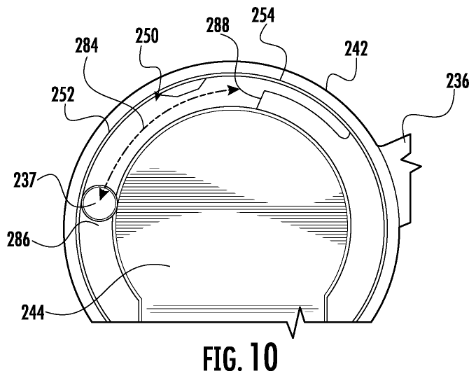

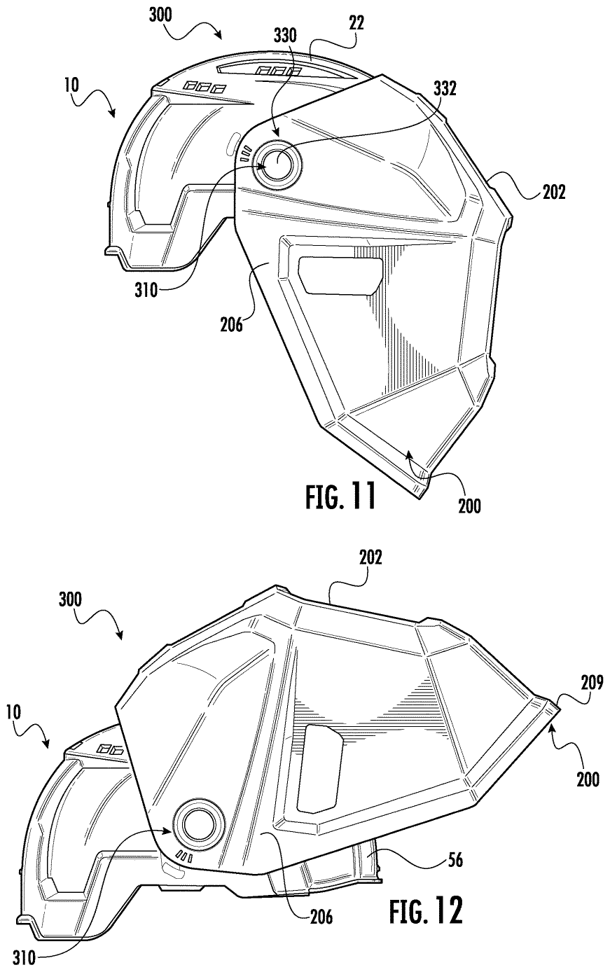

Referring to FIGS. 11-12, a side views of a protective system 300 including a work or construction helmet, shown as a hard hat 10 is shown according to an exemplary embodiment. In various embodiments, protective system 300 includes a construction helmet, shown as hard hat 10, a welding face shield 200, and a mounting component 310. Protective system 300 is substantially the same as protective system 100 except for the differences discussed herein.

As shown in FIGS. 11-12, protective system 300 is fully assembled, including a hard hat 10, 110, welding face shield 200 and mounting component(s) 310. When mounting component 310 is coupled to welding face shield 200, at least a portion of mounting component 210 is positioned outward from, such as radially or laterally outward from welding face shield body 202. In various specific embodiments, a knob 330 and/or button 332 are positioned along an exterior surface of lateral portion 206. In FIG. 11, welding face shield 200 is in a forward, in use position. As shown in FIG. 12, welding face shield 200 is in a forward, stowed position.

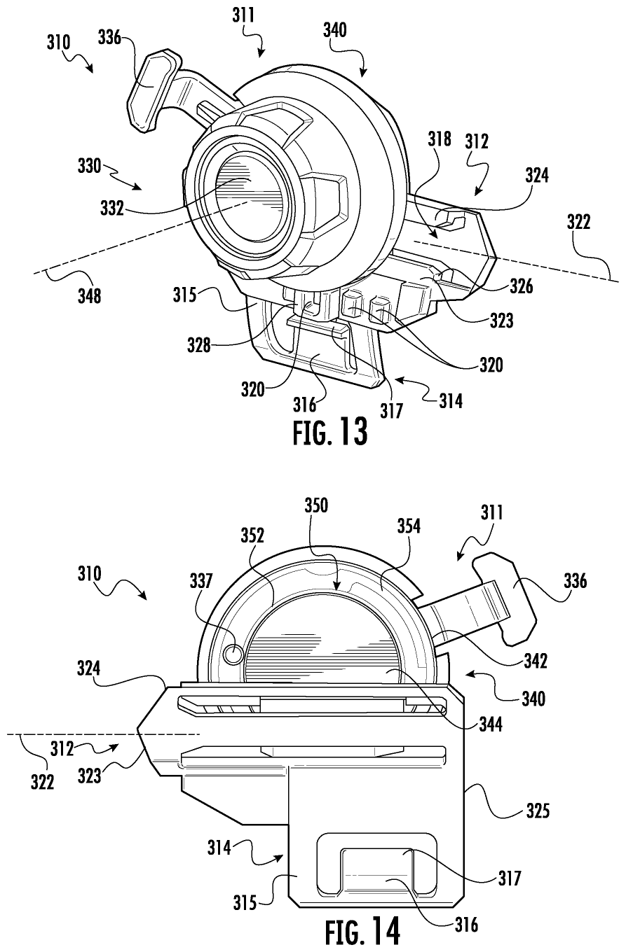

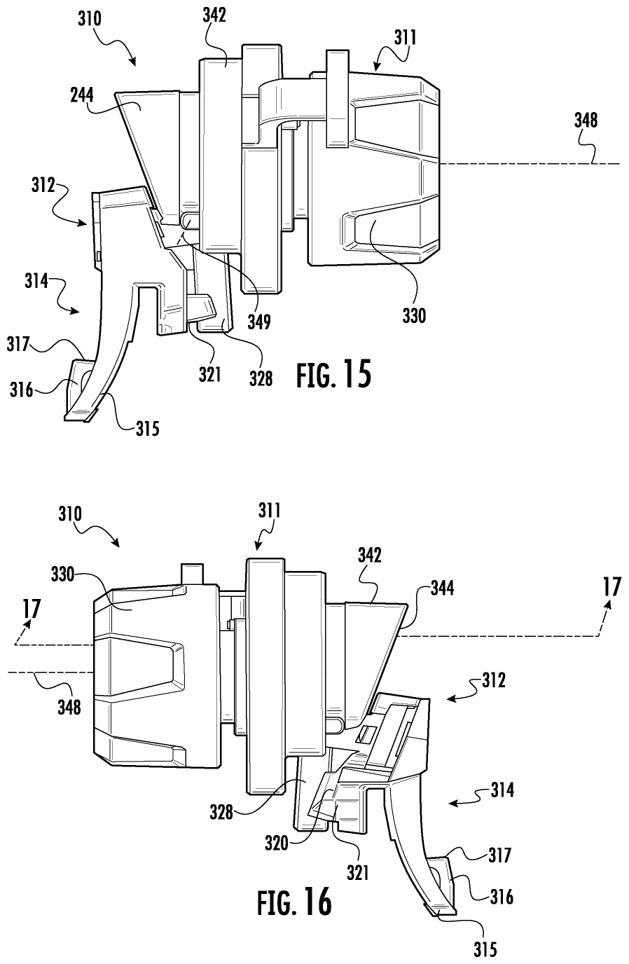

Referring to FIGS. 13-16, details of a mounting component 310 that can be utilized with protective system(s) 200, 100 are shown according to an exemplary embodiment. Mounting component 310 can be utilized with hard hat(s) 10, 110 to support an accessory such as a welding face shield 200. In specific embodiments, a pair of mounting components 310 are couplable to hard hat 10 and welding face shield 200. Similarly, mounting component 310 can be utilized with headgear and/or a suspension system to support an accessory such as welding face shield 200. Mounting component 310 is substantially the same as mounting component 210 except for the differences discussed herein.

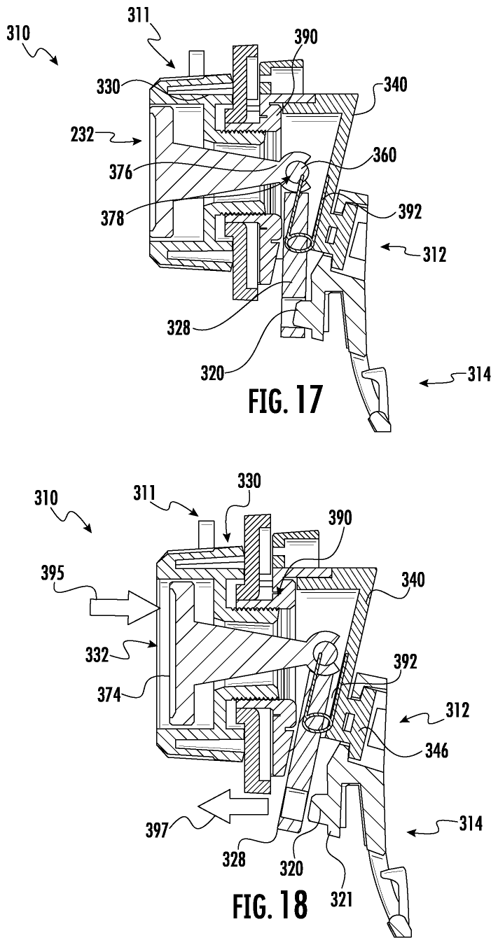

Referring to FIGS. 17-18, details of the operation of mounting component 310 are shown, according to an exemplary embodiment. In various embodiments, latch 328 is biased by a spring. In specific embodiments, latch 328 is biased by a torsion spring 392. As shown in FIG. 18, button 332 and specifically actuation surface 374 is recessed relative to an outermost surface of knob 330. When button 332 and specifically actuation surface 374 is engaged or actuated as shown by arrow 395, actuation surface 374 is moved further into knob 330 such that a depth of the recessed button 332 is increased.

When button 332 is depressed (see e.g., arrow 395), clip 378 that is coupled to latch 328 pivots the latch 328 such that latch 328 disengages or separates from the protrusion 320. As shown by arrow 397, the lower portion of latch 328 moves from being positioned along exterior surface 321 of rail 312 to a position in which latch 328 is spaced apart from rail 312 and exterior surface 321. When latch 328 is pivoted into a disengaged position (see e.g., FIG. 18), slider 340 can freely move along rail 312 to adjust the position of welding face shield 200. When button 332 is released, latch 328 will engage with the protrusion 320 (see e.g., FIG. 17) such that protrusion 320 resists fore/aft movement and the position of welding face shield 200 is secured at a point along rail 312.

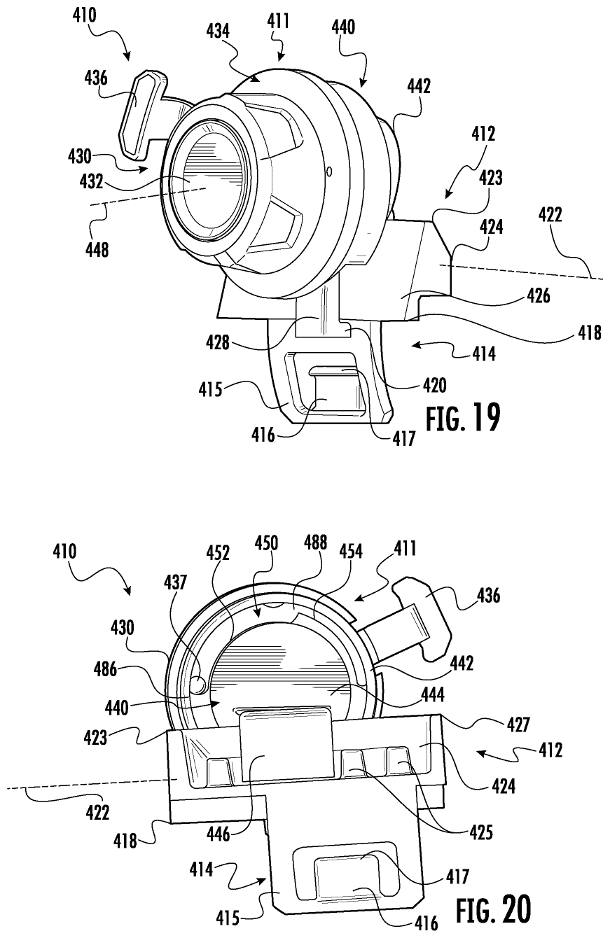

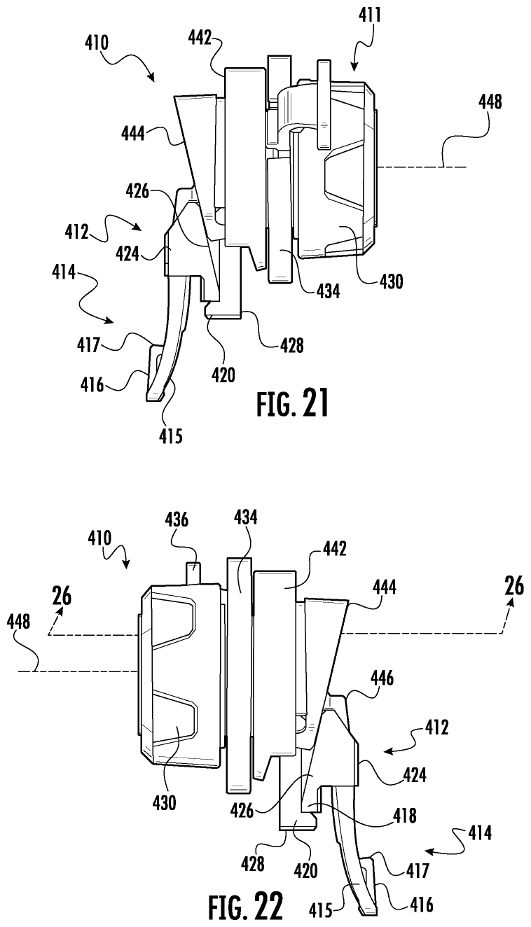

Referring to FIGS. 19-22, details of a mounting component 410 that can be utilized with protective system(s) 200, 100 are shown according to an exemplary embodiment. Mounting component 410 can be utilized with hard hat(s) 10, 110 to support an accessory such as a welding face shield 200. In specific embodiments, a pair of mounting components 410 are couplable to hard hat 10, 110 and welding face shield 200. Similarly, mounting component 410 can be utilized with headgear and/or a suspension system to support an accessory such as welding face shield 200. Mounting component 410 is substantially the same as mounting components 210, 310 except for the differences discussed herein.

In various embodiments, mounting component 410 includes a rail 412 and clip 414. In specific embodiments, rail 412 is integrally formed with clip 414. Clip 414 is configured to be coupled to hard hat 10, 110 and/or a suspension system. When clip 414 is coupled to hard hat 10, 110, clip 414 is received within a slot (see e.g., 138 in FIG. 1A) of auxiliary mounting ridge 136. In various embodiments, clip 414 includes a clip body 415 extending downward (in the orientation shown in FIG. 19) and away from rail 412. A tab, shown as resilient tab 416 extends from body 415 toward rail 412. An engagement surface, shown as an upward facing surface 417 is positioned on an uppermost portion of resilient tab 416. When clip 414 is positioned within a slot 138, resilient tab 416 and/or upward facing surface 417 engages a lower edge and/or downward facing surface of auxiliary mounting ridge 136.

As will be generally understood, rail 412 allows for movement and specific positioning of accessories such as welding face shield 200. In contrast to mounting components 210, 310 that allow for sliding movement along the respective rails 212, 312, mounting component 410 unclips and moves upward (i.e., away from rail 412) and disengages from the rail 412 before being positioned at a chosen mounting location 425. In other words, pivot joint 411 is mounted to rail 412 from the top or above.

Rail 412 includes a front surface 426 and an opposing rear surface 424. Front surface 426 extends between and connects a bottom edge 418 and a top edge 423. Bottom edge 418 is positioned between knob 430 and clip 414 when mounting component 410 is assembled. In various specific embodiments, front surface 426 is angled. In various embodiments, bottom edge 418 and/or front surface 427 projects outward relative to a top surface 427 of rail 412. In other words, bottom edge 418 acts as a projecting portion to engage latch 428 as will be discussed in greater detail below.

In specific embodiments, rear surface 424 includes one or more locating features 425 configured to engage with pivot joint 411 of mounting component 410. In a specific embodiments, the locating features 425 are protrusions extending from rear surface 424. In other embodiments, the locating features are different structures. In a specific embodiment, rail 412 includes five mounting locations 425 providing five positions for mounting component 410 and/or welding face shield 200. In other embodiments, a different number of mounting locations 425 (e.g., 3, 4, 6, 7, etc.) can be utilized. The number of mounting locations 425 corresponds to the number of positions a pivot joint 411 and specifically a latch 428 can be secured along a length of rail 412.

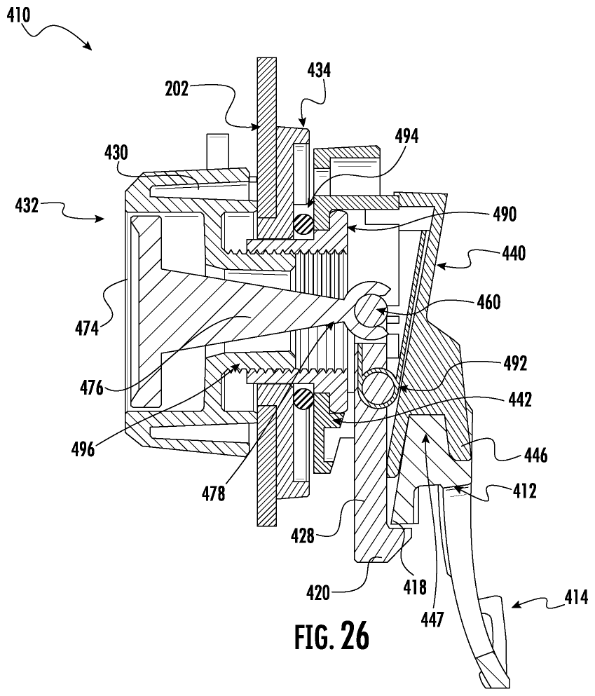

Mounting component 410 includes an arm 446 extending from rear portion 440 of pivot joint 411. In specific embodiments, arm 446 extends downward (in orientation shown in FIG. 20) toward rail 412. At least a portion of arm 446 is spaced from rear portion 440 to define a gap 447 (see e.g., FIG. 26) sized to receive and engaged rail 412. When pivot joint 411 is coupled to rail 412, arm 446 extends around an upward facing or top surface 427 of rail 412.

Pivot joint 411 is configured to allow welding face shield 200 to move and/or pivot between a up or stowed position (e.g., above brim 126 of hard hat 110) and a down or in use position in which the welding face shield 200 is positioned in front of hard hat 110 and a user's face. In various embodiments, pivot joint 411 is integral with rail 412. In specific embodiments, pivot joint 211 is integral with arm 446 and positioned above rail 412 and clip 414. Pivot joint 411 includes a latch 428 that is configured to engage rail 412 and specifically front surface 426 and/or bottom edge 418 of rail 412. In other words, in various embodiments, latch 428 engages an outwardly projecting portion of rail 412. In specific embodiments, a projection or protrusion 420 on a distal end of latch 428 engages and at least partially surrounds bottom edge 418 when latch 428 is secured to rail 412. As will be discussed in greater detail below, latch 428 is a biased latch. In specific embodiments, latch 428 is a spring biased latch.

Mounting component 410 further includes a knob 430. Knob 430 is configured to turn or rotate about axis of rotation 448. When knob 430 is rotated in a first direction, pivot joint 411 is tightened relative to rail 412 and outer shell 112 when mounting component 410 is coupled to hard hat 110. When knob 430 is rotated in the second direction, pivot joint 411 is loosened relative to rail 412 and outer shell 112 when mounting component 410 is coupled to hard hat 110.

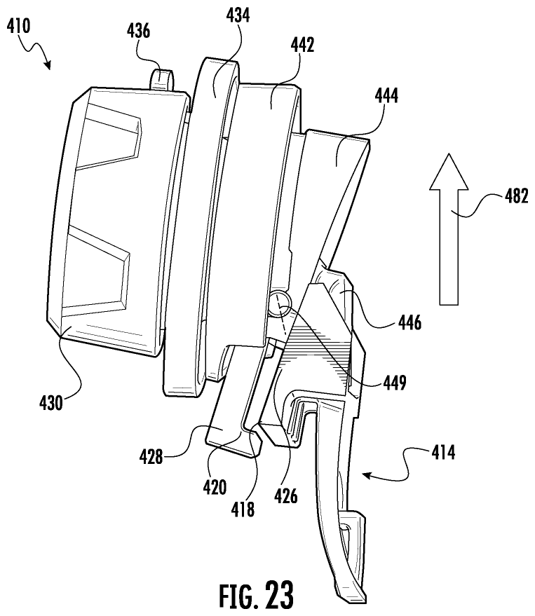

An actuator, shown as button 432 is positioned within knob 430. Button 432 is configured to engage latch 428 and move latch 428 between an engaged (see e.g., FIG. 22) and disengaged (see e.g., FIG. 23) position with rail 412 and in particular bottom edge 418. Mounting component 410 further includes an adjuster 434. Adjuster 434 is configured to engage and change an angle of the body 202 of the face shield 200. In various embodiments, adjuster 434 includes a projecting arm 436 that extends away from axis of rotation 448.

In various embodiments, mounting component 410 includes a pivot lock to secure welding face shield 200 in the up or stowed position (e.g., above brim 126 of hard hat 110) and the down or in use position (e.g., the welding face shield 200 is positioned in front of hard hat 110 and a user's face). The pivot lock includes lock 442 positioned between rear portion 440 and adjuster 434.

When rearward extending projection 437 is positioned within first section 452 of locking channel 450 and engaged with lower edge 486, welding face shield 200 cannot be moved lower. In other words, lower edge 486 acts as a lower stop for welding face shield 200 that secures welding face shield in the in use position. When a user pivots welding face shield 200 into an up or stowed position, rearward extending projection 437 moves through locking channel 450 in a direction toward second section 454 of locking channel 450. When rearward extending projection 437 reaches upper edge 488, welding face shield 200 cannot be moved higher or further up. In other words, upper edge 488 acts as an upper stop for welding face shield 200.

Referring to FIG. 23, details of the disengagement of pivot joint 411 from rail 412 are shown according to an exemplary embodiment. When an actuating surface 474 of button 432 is depressed or engaged, latch 428 moves about pivot axis 449 such that projection 420 is spaced from rail 412 and bottom edge 418. Once projection 420 is released from rail 412, pivot joint 411 can be moved upward in the direction shown by arrow 482 to release arm 446 from rail 412. A user can then choose a desired mounting location 425 and reposition pivot joint 411 along the length of rail 412. When button 432 is released, latch 428 will move back into engagement with rail 412 and in particular front surface 426 and/or bottom edge 418. Similarly, arm 446 will be repositioned around top surface 427 of rail 412.

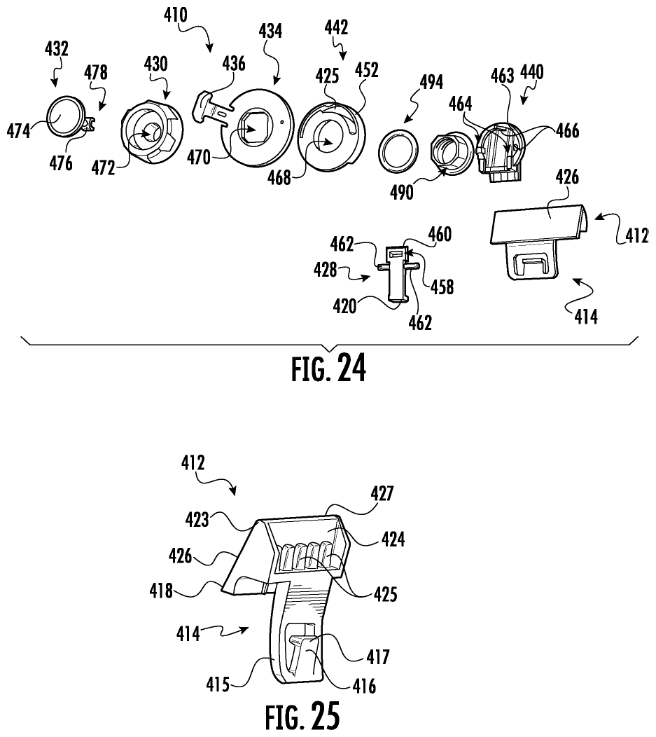

Referring to FIG. 24, an exploded view of mounting component 410 is shown according to an exemplary embodiment. A first, distal end of latch 428 includes projection 420 that is configured to engage rail 412. A second end of latch 428 includes a bore 458 that is positioned between an upper portion 460 of latch 428. Bore 458 and/or upper portion 460 are configured to engage button 432. Latch 428 further includes first and second connectors 462 that extend laterally outward from latch 428. Connectors 462 are configured to be received within a pair of recesses 464 defined along a front surface 463 of rear portion 440. The front surface 463 opposes rear surface 444 of rear portion 440. When mounting component 410 is assembled, each connector 462 is positioned in a corresponding recess 464 and movable within that recess 464 about latch pivot axis 449 (see e.g., FIG. 23) as latch 428 is pivoted to engage or disengage from rail 412.

Referring to FIG. 26, details of the operation of mounting component 410 are shown, according to an exemplary embodiment. In various embodiments, latch 428 is biased by a spring. In specific embodiments, latch 428 is biased by a torsion spring 492. In various embodiments, latch 428 is biased by one or more torsion springs 492. In a specific embodiment, latch 428 is biased by a pair of torsion springs 492. Button 432 and specifically actuation surface 474 is recessed relative to an outermost surface of knob 430. When button 432 and specifically actuation surface 474 is engaged, actuation surface 474 is moved further into knob 430 such that a depth of the recessed surface 474 is increased.

When button 432 is depressed, clip 478 that is coupled to latch 428 pivots the latch 428 such that projection 420 is removed from rail 412 and/or bottom edge 418 (see e.g., FIG. 23). The lower portion of latch 428 moves from being positioned along front surface 426 of rail 412 to a position in which latch 428 is spaced apart from rail 412 and front surface 426 allowing for repositioning of pivot joint 411.

When the protective system is assembled, body 202 of welding face shield 200 is positioned between knob 430 and adjuster 434. A sealing device, shown as O-ring 494 is positioned between adjuster 434 and lock 442. When knob 430 is turned, compression of O-ring 494 is changed. Knob 430 includes a rear connector 496. In specific embodiments, rear connector 496 includes threads. Rear connector 496 is configured to engage a nut shown as flanged nut 490. Flanged nut 490 is positioned within lock 442.

It should be understood that the figures illustrate the exemplary embodiments in detail, and it should be understood that the present application is not limited to the details or methodology set forth in the description or illustrated in the figures. It should also be understood that the terminology is for the purpose of description only and should not be regarded as limiting.

Further modifications and alternative embodiments of various aspects of the disclosure will be apparent to those skilled in the art in view of this description. Accordingly, this description is to be construed as illustrative only. The construction and arrangements, shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present disclosure.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred. In addition, as used herein, the article “a” is intended to include one or more component or element, and is not intended to be construed as meaning only one.

For purposes of this disclosure, the term “coupled” means the joining of two components directly or indirectly to one another. Such joining may be stationary in nature or movable in nature. Such joining may be achieved with the two members and any additional intermediate members being integrally formed as a single unitary body with one another or with the two members or the two members and any additional member being attached to one another. Such joining may be permanent in nature or alternatively may be removable or releasable in nature. As used herein, “rigidly coupled” refers to two components being coupled in a manner such that the components move together in a fixed positional relationship when acted upon by a force.

While the current application recites particular combinations of features in the claims appended hereto, various embodiments of the invention relate to any combination of any of the features described herein whether or not such combination is currently claimed, and any such combination of features may be claimed in this or future applications. Any of the features, elements, or components of any of the exemplary embodiments discussed above may be used alone or in combination with any of the features, elements, or components of any of the other embodiments discussed above.

In various exemplary embodiments, the relative dimensions, including angles, lengths and radii, as shown in the Figures are to scale. Actual measurements of the Figures will disclose relative dimensions, angles and proportions of the various exemplary embodiments. Various exemplary embodiments extend to various ranges around the absolute and relative dimensions, angles and proportions that may be determined from the Figures. Various exemplary embodiments include any combination of one or more relative dimensions or angles that may be determined from the Figures. Further, actual dimensions not expressly set out in this description can be determined by using the ratios of dimensions measured in the Figures in combination with the express dimensions set out in this description.

What is claimed is:

1. A protective system comprising:
a face shield;
a hard hat or suspension system configured to support the face shield;
a mounting component configured to couple to the hard hat or suspension system and support the face shield relative to the hard hat or suspension system, the mounting component comprising:
a rail defining a longitudinal axis, the rail comprising one or more projecting portions;
a pivot joint configured to move the face shield between a stowed position and an in-use position, the pivot joint comprising:
a slider configured to move along the longitudinal axis of the rail to adjust a position of the face shield;
a button connected to a latch;
wherein, when the button is actuated, the latch is pivoted to a position spaced from the one or more projecting portions of the rail such that the position of the slider is movable along the longitudinal axis of the rail; and
wherein, when the button is not engaged, the latch engages the one or more projecting portions to resist movement of the slider along the longitudinal axis of the rail.

2. The protective system of claim 1, wherein the mounting component further comprises a knob positioned around the button, wherein the knob is configured to rotate about an axis of rotation.

3. The protective system of claim 2, wherein, when the knob is rotated in a first direction, the pivot joint is tightened relative to the rail such that a force required to lift the face shield is increased, and wherein, when the knob is rotated in a second direction that is opposite the first direction, the pivot joint is loosened relative to the rail such that a force required to lift the face shield is decreased.

4. The protective system of claim 2, wherein the mounting component further comprises an adjuster positioned between the slider and the knob, wherein the adjuster is configured to engage the face shield and change an angle of the face shield relative to the hard hat or suspension system.

5. The protective system of claim 4, wherein the adjuster further comprises a projection extending away from the knob, the projection positioned within a locking channel.

6. The protective system of claim 5, wherein the locking channel comprises a lower edge and an upper edge, and wherein the projection of the adjuster interfaces against the lower edge of the locking channel when the face shield is the in-use position.

7. The protective system of claim 6, wherein the projection of the adjuster interfaces against the upper edge of the locking channel when the face shield is in the stowed position.

8. The protective system of claim 2, wherein an actuation surface of the button is recessed relative to an outermost surface of the knob.

9. The protective system of claim 1, wherein at least a portion of the mounting component is positioned laterally outward from the face shield.

10. A protective system comprising:
a face shield;
a hard hat; and
a mounting component configured to support the face shield relative to the hard hat, the mounting component comprising:
a rail defining a longitudinal axis, the rail comprising one or more protrusions; and
a pivot joint configured to move the face shield between a stowed position and an in-use position, the pivot joint comprising:
a latch;
wherein the latch is pivotable between a disengaged position in which the latch is spaced from the one or more protrusions of the rail and an engaged position in which the latch interfaces with the one or more protrusions to secure the pivot joint to the rail.

11. The protective system of claim 10, further comprising a clip integrally formed with the rail, the clip configured to couple to the hard hat via a slot on the hard hat.

12. The protective system of claim 11, wherein the clip comprises a body extending away from the rail and a resilient tab extending from the body toward the rail, wherein an upper surface of the resilient tab engages the hard hat to support the rail relative to the hard hat.

13. The protective system of claim 10, wherein the pivot joint is integral with a slider configured to move the pivot joint along a longitudinal axis of the rail, and wherein the slider is positioned above the rail.

14. The protective system of claim 10, further comprising one or more torsion springs that bias the latch into engagement with the rail.

15. A protective system comprising:
a face shield;
a hard hat; and
a mounting component configured to support the face shield relative to the hard hat, the mounting component comprising:
a rail defining a length along a longitudinal axis, the rail comprising:
at least one protrusion extending outward from the rail; and
a clip configured to engage a slot defined in the hard hat; and
a pivot joint configured to move the face shield between a stowed position and an in use position, the pivot joint comprising:
a button; and
a latch connected to the button and configured to be actuated by the button;
wherein the latch is actuated between a first position in which the latch is disengaged from the at least one protrusion such that the pivot joint is adjustable and a second position in which the latch is engaged with the at least one protrusion such that the pivot joint is secured.

16. The protective system of claim 15, wherein the at least one protrusion is a plurality of protrusions, and wherein each of the plurality of protrusions corresponds to a position at which the pivot joint can be positioned along the length of the rail.

17. The protective system of claim 15, wherein the latch comprises:
a first end coupled to the button; and
a second end opposite the first end, the second end comprising a bore.

18. The protective system of claim 17, wherein the bore at the second end of the latch is positioned around a perimeter of the at least one protrusion when the latch is in the second position.

19. The protective system of claim 15, wherein the mounting component further comprises an adjuster, wherein the adjuster is configured to change an angle of the face shield relative to the hard hat.

20. The protective system of claim 19, wherein the adjuster comprises an arm extending radially outward, and wherein the arm couples to the face shield.
